(19) United States
(12) Patent Application Publication
(10) Pub. No.: US 2023/0290464 A1
(43) Pub. Date: Sep. 14, 2023

(54) SYSTEMS AND METHODS FOR VERIFYING PRESCRIPTIONS

(71) Applicant: Change Healthcare Holdings, LLC, Nashville, TN (US)

(72) Inventor: Benjamin Joseph Diatto, Aledo, TX (US)

(21) Appl. No.: 17/691,341

(22) Filed: Mar. 10, 2022

(51) Int. Cl. G16H 20/10; G16H 70/40

(52) U.S. Cl. CPC G16H 20/10; G16H 70/40

(57) ABSTRACT

In an embodiment, systems and method for detecting errors in prescriptions are provided. When a prescription for a medication is received by a prescription fulfillment system, the instructions associated with the prescription are analyzed to determine where the prescription for the medication includes a mistake. The analysis may be performed based on information collected from a plurality of prescriptions for the same medication that are known to not include any mistakes. If a prescription is determined to possibly include a mistake one or more actions may be taken including notifying a pharmacist, notifying the medical provider associated with the prescription, notifying the patient, and in some circumstances, rejecting the prescription. In some embodiments, the analysis may be performed by a model trained using the plurality of prescriptions known to not include any mistakes, and a plurality of prescriptions known to include some mistakes.

### BACKGROUND

Medical providers, such as doctors, write prescriptions for medications for patients under their care. These prescriptions are then fulfilled by pharmacists for the patients. Many medications can be dangerous if not taken correctly according to instructions. These instructions may include the recommended dosage, frequency of administration (e.g., twice a day), and conditions under which to take the medication (e.g., with water, on an empty stomach, after eating, before bed, and in the morning). Other instructions may include avoiding certain foods while taking the medication (e.g., no grapefruit, and no alcohol), and avoiding certain activities (e.g., no operating heavy machinery).

While medical providers are careful when writing prescriptions, mistakes are made. Pharmacists, when fulfilling prescriptions, may be able to detect and correct some of these errors, but given the volume of prescriptions some mistakes may not be detected. Accordingly, there is a need to automatically detect or flag prescription mistakes before they provided to patients.

### SUMMARY

In an embodiment, systems and methods for detecting errors in prescriptions are provided. When a prescription for a medication is received by a prescription fulfillment system, the instructions associated with the prescription are analyzed to determine where the prescription for the medication includes a mistake. The analysis may be performed based on information collected from a plurality of prescriptions for the same medication that are known to not include any mistakes. If a prescription is determined to possibly include a mistake one or more actions may be taken including notifying a pharmacist, notifying the medical provider associated with the prescription, notifying the patient, and in some circumstances, rejecting the prescription. In some embodiments, the analysis may be performed by a model trained using the plurality of prescriptions known to not include any mistakes, and a plurality of prescriptions known to include some mistakes.

The systems and methods described herein provide the following advantages. First, identifying prescription mistakes and errors can prevent possible injury to patients including death. Second, by automating the prescription mistake detection process, pharmacists can spend more time fulfilling prescriptions and interacting with patients instead of looking for mistakes in prescriptions.

Additional advantages of the invention will be set forth in part in the description which follows, and in part will be obvious from the description, or may be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### BRIEF DESCRIPTION OF THE DRAWINGS

The accompanying figures, which are incorporated herein and form part of the specification, illustrate a prescription mistake prevention system and method. Together with the description, the figures further serve to explain the principles of the prescription mistake prevention system and method described herein and thereby enable a person skilled in the pertinent art to make and use the prescription mistake prevention system and method.

FIG. 1 is an example environment for fulfilling prescriptions;

FIG. 2 is an illustration of an example method for training a model to identify mistakes in prescriptions;

FIG. 3 is an illustration of an example method for generating rules to identify mistakes in prescriptions;

FIG. 4 is an illustration of an example method for detecting mistakes in prescriptions; and

FIG. 5 shows an exemplary computing environment in which example embodiments and aspects may be implemented.

### DETAILED DESCRIPTION

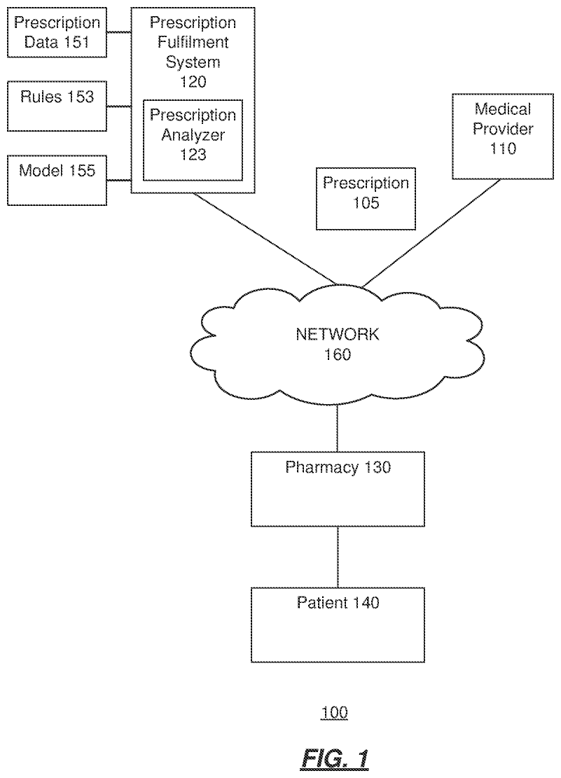

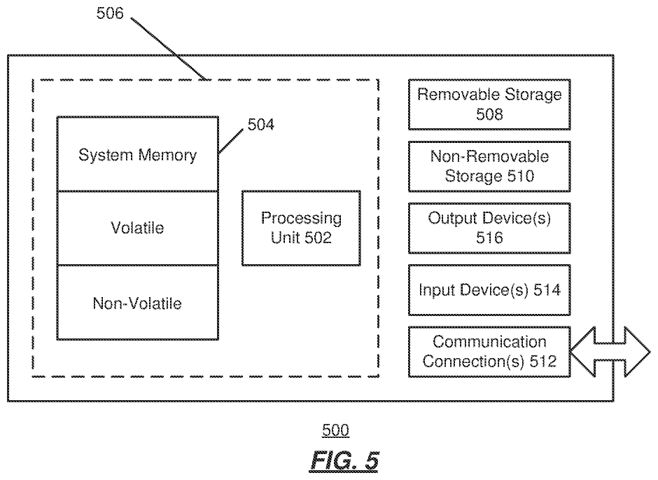

FIG. 1 is an example environment for fulfilling prescriptions. As shown, the environment 100 may include a prescription fulfillment system 120, one or more medical providers 110, and one or more pharmacies 130 in communication through a network 160. The network 160 may include a combination of private networks (e.g., LANs) and public networks (e.g., the Internet). Each of the prescription fulfillment system 120, the medical provider 110, and the pharmacy 130 may be partially implemented using one or more general purpose computing devices such as the computing device 500 illustrated in FIG. 5.

In some embodiments, the prescription fulfillment system 120 may receive prescriptions 105 from one or more medical providers 110 through the network 160. The medical providers 110 may include doctors, nurse practitioners, medical practices, and hospitals, for example.

In general, each prescription 105 may identify a medication and a patient 140. Each prescription 105 may further include instructions for the patient 140 on how to take the medication. The instructions may include, but are not limited to, instructions related to dosage, frequency, timing, conditions under which the medication should be taken (e.g., after eating or before eating), and activities or foods that should le be avoided while taking the medication (e.g., no alcohol, no grapefruit, and no driving or operating heavy machinery).

The medical provider 110 may provide the prescription 105 to the prescription fulfillment system 120 through the network 160 in an electronic format. In some embodiments, the prescription fulfillment system 120 may provide a user interface through which the medical provider 110 may generate the prescription 105 including the medication, patient 140, and instructions. Alternately or additionally, the prescription fulfillment system 120 may provide or expose an application programing interface (API) that may allow the medical providers 110 to use applications of choice to generate and provide the prescriptions 105.

The prescription fulfillment system 120 may receive the prescriptions 105, may store or record the prescriptions 105, and may forward them to one or more pharmacies 130 for fulfillment via the network 160. The pharmacy 130 that receives the prescription 105 may have been specified by one or both of the medical provider 110 or the patient 140.

The pharmacy 130 may receive the prescription 105 from the prescription fulfillment system 120. The pharmacy 130 may receive the prescription 105 using a user interface provided by the prescription fulfilment system 120. Alternatively, or additionally, the pharmacy 130 may receive the prescription 105 using an existing prescription management system or other application used by the pharmacy through an API exposed by the prescription fulfilment system 120.

A pharmacist associated with the pharmacy 130 may view the received prescription 105 and may fulfil the prescription 105 for the patient 140. As part of the fulfilment process, the pharmacist may print out or otherwise provide instructions to the patient 140 that may be based on, or the same as, the instructions associated with the prescription that were provided by the medical provider 110 with the prescription 105.

As described above, a problem associated with prescriptions 105 are mistakes. These mistakes may be associated with the instructions of the prescription 105 and may include mistakes such as mistakes related to the dosage (e.g., the dosage for the medication may be too high or too low for the patient 140), mistakes related to the frequency (e.g., the patient is instructed to take the medication more or less often than is recommended), and mistakes related to how or when the medication should be taken (e.g., the medication should be taken with food and it is not listed on the instructions, or the medication should be taken before bed and the instructions list the morning). Other types of mistakes may be supported.

In order to identify and possibly correct mistakes in prescriptions 105, the prescription fulfillment system 120 may further include a prescription analyzer 123 that identifies possible mistakes or errors in prescriptions 105, and alerts one or more of the associated medical providers 110, the associated pharmacy 130, and the associated patient 140 regarding the mistake. Depending on the embodiment, the prescription analyzer 123 may identify mistakes (if any) in the prescription 105 after it is received from the medical provider 110, or after it has been sent to the pharmacy 130.

The prescription analyzer 123 may receive prescription data 151 and may use the received prescription data 151 to identify mistakes in the prescriptions 105. The prescription data 151 may include previously received prescriptions 105, including instructions, received from various medical providers 110 for a variety of different medications. The prescriptions 105 in the prescription data 151 may have been verified to include no mistakes. For example, one or more doctors or medical providers 110 may have reviewed the prescriptions 105 in the prescription data 151 and verified that they include no mistakes.

In some embodiments, prescription analyzer 123 identify mistakes in a received prescription 105 for a medication by comparing the received prescription 105 with the stored prescriptions 105 in the prescription data 151 for the same medication. Differences in the received prescription 105 and the stored prescriptions 105 may be flagged as possible mistakes.

In some embodiments, the prescription analyzer 123 may generate one or more rules 153 for each medication based on the prescriptions 105 in the prescription data 151 for the medication. The rules 153 for each medication may be based on the instructions associated with the prescriptions 105 in the prescription data 151. The rules 153 for a medication may include a rule 153 for the dosage, a rule for the frequency of the dosage, and a rule for the timing of the dosage. In some embodiments, each rule 153 may be a value or range of values that is learned from the prescriptions 105 in the prescription data 151. For example, if the dosage for a particular medication is usually between 5 mg and 20 mg in the prescriptions 105 of the prescription data 151, the prescription analyzer 123 may generate a rule 153 for the medication that the dosage is between 5 mg and 20 mg.

When the prescription analyzer 123 receives a prescription 105, the prescription analyzer 123 may retrieve the rules 153 generated for the medication associated with the prescription 105 and may compare each rule 153 to the prescription 105. Any rule 153 that is violated by the prescription 105 may indicate that there is a mistake in the prescription 105.

In some embodiments, rather than using rules 153, the prescription analyzer 123 may train a model 155 to identify mistakes in prescriptions 105 using the prescriptions 105 and associated instructions in the prescription data 151. The model 155 may receive as an input a prescription 105 and instructions and may output a probability or confidence that the prescription 105 includes a particular mistake. If the probability for a mistake is above a threshold probability than the prescription analyzer 123 may determine that the prescription 105 has the particular mistake.

The prescription analyzer 123 may train a model 155 for each medication or may train a single model 155 to identify mistakes for multiple medications. In some embodiments, the prescription analyzer 123 may train the model 155 using machine learning. In machine learning, training data may be used to train a model 155. The training data may include prescriptions 105 that are known to not include any mistakes and prescriptions 105 that are known to include certain mistakes. The training data may be labeled training data and may be labeled by one or more medical providers 110. Any method for training a model 155 may be used.

The prescription analyzer 123, upon detecting a mistake in a prescription 105 (using a model 155 or one or more rules 153), may take one or more more actions with respect to the prescription 105. In some embodiments, the prescription analyzer 123 may flag the mistake in the prescription so that when the prescription 105 is fulfilled by the pharmacist at the pharmacy 130, the pharmacist is made aware of the mistake. The pharmacist may then take one or more actions such as notifying the patient 140 or medical provider 110 that wrote the prescription 105. When the prescription 105 is fulfilled for the patient 140, the detected mistake may be indicated to the patient 140 along with the medication instructions. Alternatively, when a mistake is detected, the prescription analyzer 123 may prevent the prescription 105 from being fulfilled and may ask the medical provider 110 to submit a new prescription 105.

In some embodiments, when a mistake is detected the prescription analyzer 123 may notify the medical provider 110 of the detected mistake. The medical provider 110 may then either correct the mistake or may override the prescription analyzer 123 and certify that the mistake is not a mistake. In the event that the medical provider 110 determines that the mistake was correct, the prescription analyzer 123 may update the model 155 and/or rules 153 so that the mistake is not identified in the future.

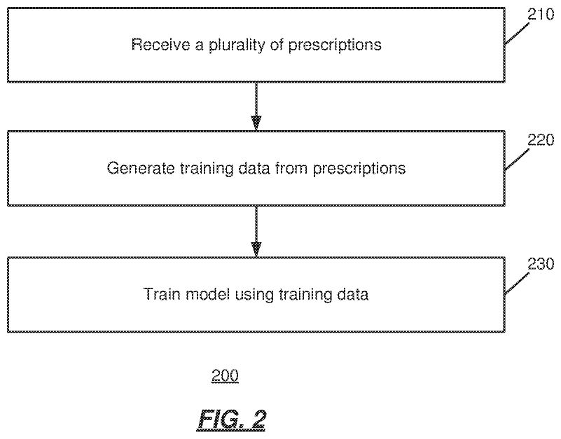

FIG. 2 is an illustration of an example method 200 for training a model to identify mistakes in prescriptions. The method 200 may be implemented by the prescription analyzer 123 of the prescription fulfillment system 120.

At 210, a plurality of prescriptions is received. The plurality of prescriptions may be received by the prescription analyzer 123 as the prescription data 151. The prescriptions 105 may be previously received prescriptions 105 for patients 140 from medical providers 110. Each prescription may include instructions and may be associated with a particular medication.

At 220, training data is generated from the prescriptions. The training data may be labeled training data and may include prescriptions 105 that are labeled as having no mistakes, and prescriptions 105 that are labeled as having one or more particular mistakes (e.g., incorrect dosage, or incorrect timing, or incorrect frequency). The labels may be generated by one or more human reviewers such as doctors or other medical providers 110 reviewing the prescriptions 105.

At 230, a model is trained using the training data. The model 155 may be a machine learning model and may be trained using the labeled training data. The model 155 may receive a prescription 105 including instructions and may output a probability that the prescription 105 has a particular mistake. Depending on the embodiment, there may be a single model 155 or multiple models 155. Where there are multiple models 155, each model 155 may be trained to identify a particular type of mistake or may be trained for prescriptions 105 for a particular type of medication. For example, a model 155 may be trained to identify mistakes in prescriptions 105 for the medication Atorvastatin and another model 155 may be trained to identify mistakes in prescriptions 105 for the medication Lisinopril.

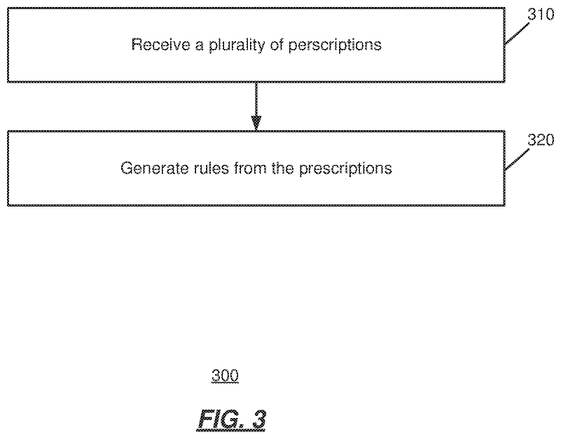

FIG. 3 is an illustration of an example method for generating rules to identify mistakes in prescriptions. The method 300 may be implemented by the prescription analyzer 123 of the prescription fulfillment system 120.

At 310, a plurality of prescriptions is received. The plurality of prescriptions may be received by the prescription analyzer 123 as the prescription data 151. The prescriptions may be previously received prescriptions for patients 140 from medical providers 110. Each prescription 105 may include instructions and may be associated with a particular medication.

At 320, one or more rules are generated from the prescriptions. The one or more rules 153 may be generated by the prescription analyzer 123 from the prescription data 151. Depending on the embodiment, the prescription analyzer 123 may generate the rules 153 by processing the prescriptions 105, including instructions, to identify characteristics and qualities associated with correct prescriptions 105. Each rule 153 may be associated with a different medication.

For example, the prescription analyzer 123 may identify a range of dosages associated with the prescriptions 105 and may generate a rule 153 that codifies the range of dosages. As another example, the prescription analyzer 105 may determine that most of the prescriptions 105 for a medication say that the medication should be taken before bedtime. Accordingly, the prescription analyzer 105 may generate a rule 153 for the medication that the prescription 105 should indicate that the medication is taken before bedtime.

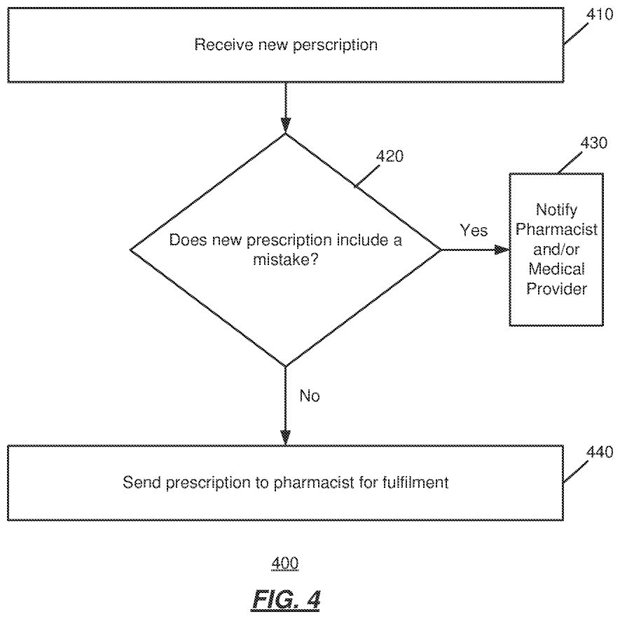

FIG. 4 is an illustration of an example method for detecting mistakes in prescriptions. The method 400 may be implemented by the prescription analyzer 123.

At 410, a new prescription is received. The new prescription 105 may be received by the prescription fulfillment system 120. The prescription 105 may be received from a medical provider 110 and may be a prescription 105 for a medication to be taken by a patient 140. The prescription 105 may include instructions.

At 420, whether the new prescription includes a mistake is determined. The determination may be made by the prescription analyzer 123 of the prescription fulfillment system 120. In some embodiments, the prescription analyzer 123 may determine whether the new prescription 105 includes any mistakes using a model 155 trained to identify mistakes in prescriptions 105 for the medication associated with the new prescription 105. Alternatively, the prescription analyzer 123 may determine whether the new prescription 105 includes any mistakes using one or more rules 153 generated based on previously received prescriptions 105 for the medication. If the prescription analyzer 123 determines any mistakes, then the method 400 may continue at 430. Else, no mistakes are determined and the method 400 may continue at 440.

At 430, a pharmacists or medical provider are notified. The pharmacists or medical provider 110 may be notified by the prescription analyzer 123. For example, the prescription analyzer 123 may generate and send an electronic message that the prescription may include one or more mistakes. The message may indicate the one or more mistakes that were identified and may indicate how the mistake may be corrected. In some embodiments, the prescription analyzer 123 may allow the pharmacy 130 to fulfill the prescription 105 after one or both of the medical provider 110 and the pharmacist corrects the mistake or determines that there was no mistake and the instructions were intentional.

At 440, the prescription is sent to the pharmacy 130 for fulfillment. The prescription 105 may be sent by the prescription analyzer 123 to the pharmacy 130 through the network 160. The prescription 105 may indicate that the instructions of the prescription 105 were analyzed by the prescription analyzer 123 and no mistakes were detected.

FIG. 5 shows an exemplary computing environment in which example embodiments and aspects may be implemented. The computing device environment is only one example of a suitable computing environment and is not intended to suggest any limitation as to the scope of use or functionality.

Numerous other general purpose or special purpose computing devices environments or configurations may be used. Examples of well-known computing devices, environments, and/or configurations that may be suitable for use include, but are not limited to, personal computers, server computers, handheld or laptop devices, multiprocessor systems, microprocessor-based systems, network personal computers (PCs), minicomputers, mainframe computers, embedded systems, distributed computing environments that include any of the above systems or devices, and the like.

Computer-executable instructions, such as program modules, being executed by a computer may be used. Generally, program modules include routines, programs, objects, components, data structures, etc. that perform particular tasks or implement particular abstract data types. Distributed computing environments may be used where tasks are performed by remote processing devices that are linked through a communications network or other data transmission medium. In a distributed computing environment, program modules and other data may be located in both local and remote computer storage media including memory storage devices.

With reference to FIG. 5, an exemplary system for implementing aspects described herein includes a computing device, such as computing device 500. In its most basic configuration, computing device 500 typically includes at least one processing unit 502 and memory 504. Depending on the exact configuration and type of computing device, memory 504 may be volatile (such as random access memory (RAM)), non-volatile (such as read-only memory (ROM), flash memory, etc.), or some combination of the two. This most basic configuration is illustrated in FIG. 5 by dashed line 506.

Computing device 500 may have additional features/functionality. For example, computing device 500 may include additional storage (removable and/or non-removable) including, but not limited to, magnetic or optical disks or tape. Such additional storage is illustrated in FIG. 5 by removable storage 508 and non-removable storage 510.

Computing device 500 typically includes a variety of computer readable media. Computer readable media can be any available media that can be accessed by the device 500 and includes both volatile and non-volatile media, removable and non-removable media.

Computer storage media include volatile and non-volatile, and removable and non-removable media implemented in any method or technology for storage of information such as computer readable instructions, data structures, program modules or other data. Memory 504, removable storage 508, and non-removable storage 510 are all examples of computer storage media. Computer storage media include, but are not limited to, RAM, ROM, electrically erasable program read-only memory (EEPROM), flash memory or other memory technology, CD-ROM, digital versatile disks (DVD) or other optical storage, magnetic cassettes, magnetic tape, magnetic disk storage or other magnetic storage devices, or any other medium which can be used to store the desired information and which can be accessed by computing device 500. Any such computer storage media may be part of computing device 500.

Computing device 500 may contain communication connection(s) 512 that allow the device to communicate with other devices. Computing device 500 may also have input device(s) 514 such as a keyboard, mouse, pen, voice input device, touch input device, etc. Output device(s) 516 such as a display, speakers, printer, etc. may also be included. All these devices are well known in the art and need not be discussed at length here.

It should be understood that the various techniques described herein may be implemented in connection with hardware components or software components or, where appropriate, with a combination of both. Illustrative types of hardware components that can be used include Field-programmable Gate Arrays (FPGAs), Application-specific Integrated Circuits (ASICs), Application-specific Standard Products (ASSPs), System-on-a-chip systems (SOCs), Complex Programmable Logic Devices (CPLDs), etc. The methods and apparatus of the presently disclosed subject matter, or certain aspects or portions thereof, may take the form of program code (i.e., instructions) embodied in tangible media, such as floppy diskettes, CD-ROMs, hard drives, or any other machine-readable storage medium where, when the program code is loaded into and executed by a machine, such as a computer, the machine becomes an apparatus for practicing the presently disclosed subject matter.

Although exemplary implementations may refer to utilizing aspects of the presently disclosed subject matter in the context of one or more stand-alone computer systems, the subject matter is not so limited, but rather may be implemented in connection with any computing environment, such as a network or distributed computing environment. Still further, aspects of the presently disclosed subject matter may be implemented in or across a plurality of processing chips or devices, and storage may similarly be affected across a plurality of devices. Such devices might include personal computers, network servers, and handheld devices, for example.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

What is claimed is:

1. A method comprising:
receiving a new prescription for a medication by a computing device, wherein the prescription includes instructions;
based on a plurality of previously fulfilled prescriptions associated with the medication, determining whether the new prescription includes a mistake by the computing device; and
if it is determined that the new prescription includes the mistake, notifying a pharmacist associated with the prescription about the mistake.

2. The method of claim 1, further comprising:
if it is determined that the new prescription includes the mistake, notifying a medical provider associated with the new prescription about the mistake.

3. The method of claim 1, further comprising:
if it is determined that the new prescription includes the mistake, preventing the pharmacist associated with the new prescription from fulfilling the new prescription.

4. The method of claim 1, wherein if it is determined that the new prescription does not include the mistake, providing the new prescription to the pharmacist for fulfillment.

5. The method of claim 1, wherein based on the plurality of previously fulfilled prescriptions associated with the medication, determining whether the new prescription includes the mistake comprises:
analyzing the plurality of previously fulfilled prescriptions associated with the medication to generate one or more rules for the medication; and
determining whether the prescription includes the mistake using the one or more rules.

6. The method of claim 1, wherein based on the plurality of previously fulfilled prescriptions associated with the medication, determining whether the new prescription includes the mistake comprises:
using the plurality of previously fulfilled prescriptions associated with the medication to train a model to identify one or more mistakes including the mistake; and
determining whether the new prescription includes the mistake using the model.

7. The method of claim 1, wherein the mistake includes one or more of a dosage mistake, a frequency mistake, and a timing mistake.

8. A system comprising:
at least one computing device; and
a non-transitory computer-readable medium with computer-executable instructions stored thereon that when executed by the at least one computing device cause the at least one computing device to:
receive a plurality of previously fulfilled prescriptions for a medication;
receive a new prescription for the medication, wherein the received new prescription includes instructions;
based on the plurality of prescriptions associated with the medication, determining whether the received new prescription includes a mistake; and
if it is determined that the new prescription includes the mistake, notify a pharmacist associated with the received prescription about the mistake.

9. The system of claim 8, further comprising computer-executable instructions stored thereon that when executed by the at least one computing device cause the at least one computing device to:
if it is determined that the new prescription includes the mistake, notify a medical provider associated with the new prescription about the mistake.

10. The system of claim 8, further comprising computer-executable instructions stored thereon that when executed by the at least one computing device cause the at least one computing device to:
if it is determined that the new prescription includes the mistake, prevent the pharmacist associated with the prescription from fulfilling the prescription.

11. The system of claim 8, wherein if it is determined that the new prescription does not include the mistake, providing the prescription to the pharmacist for fulfillment.

12. The system of claim 8, wherein based on the plurality of fulfilled prescriptions associated with the medication, determining whether the new prescription includes the mistake comprises:
analyzing the plurality of previously fulfilled prescriptions associated with the medication to generate one or more rules for the medication; and
determining whether the new prescription includes the mistake using the one or more rules.

13. The system of claim 8, wherein based on the plurality of previously fulfilled prescriptions associated with the medication, determining whether the new prescription includes the mistake comprises:
using the plurality of previously fulfilled prescriptions associated with the medication to train a model to identify one or more mistakes including the mistake; and
determining whether the new prescription includes the mistake using the model.

14. The system of claim 8, wherein the mistake includes one or more of a dosage mistake, a frequency mistake, and a timing mistake.

15. A non-transitory computer-readable medium with computer-executable instructions stored thereon that when executed by at least one computing device cause the at least one computing device to:
receive a plurality of previously fulfilled prescriptions for a medication;
receive a new prescription for the medication, wherein the received new prescription includes instructions;
based on the plurality of prescriptions associated with the medication, determining whether the received new prescription includes a mistake; and
if it is determined that the new prescription includes the mistake, notify a pharmacist associated with the received prescription about the mistake.

16. The non-transitory computer-readable medium of claim 15, further comprising computer-executable instructions stored thereon that when executed by the at least one computing device cause the at least one computing device to:
if it is determined that the new prescription includes the mistake, notify a medical provider associated with the new prescription about the mistake.

17. The non-transitory computer-readable medium of claim 15, further comprising computer-executable instructions stored thereon that when executed by the at least one computing device cause the at least one computing device to:
if it is determined that the new prescription includes the mistake, prevent the pharmacist associated with the prescription from fulfilling the prescription.

18. The non-transitory computer-readable medium of claim 15, wherein if it is determined that the new prescription does not include the mistake, providing the prescription to the pharmacist for fulfillment.

19. The non-transitory computer-readable medium of claim 15, wherein based on the plurality of fulfilled prescriptions associated with the medication, determining whether the new prescription includes the mistake comprises:
analyzing the plurality of previously fulfilled prescriptions associated with the medication to generate one or more rules for the medication; and
determining whether the new prescription includes the mistake using the one or more rules.

20. The non-transitory computer-readable medium of claim 15, wherein based on the plurality of previously fulfilled prescriptions associated with the medication, determining whether the new prescription includes the mistake comprises:
using the plurality of previously fulfilled prescriptions associated with the medication to train a model to identify one or more mistakes including the mistake; and
determining whether the new prescription includes the mistake using the model.
